(19) United States
(12) Patent Application Publication
(10) Pub. No.: US 2025/0135197 A1
(43) Pub. Date: May 1, 2025

(54) TRANSDUCER ARRAY WITH ANISOTROPIC MATERIAL LAYER HAVING A SHAPE THAT CONTOURS TO A SUBJECT'S BODY

(71) Applicant: Novocure GmbH, Root D4 (CH)

(72) Inventors: Darja WENDEL, Lisbon (PT); Woonghee HAN, San Francisco, CA (US)

(73) Assignee: Novocure GmbH, Root D4 (CH)

(21) Appl. No.: 19/007,003

(22) Filed: Dec. 31, 2024

Related U.S. Application Data
(63) Continuation-in-part of application No. 18/080,306, filed on Dec. 13, 2022.
(60) Provisional application No. 63/289,523, filed on Dec. 14, 2021.

(51) Int. Cl. A61N 1/36; A61N 1/04; A61N 1/40

(52) U.S. Cl. CPC A61N 1/36002; A61N 1/0476; A61N 1/0496; A61N 1/40

(57) ABSTRACT

A transducer apparatus for delivering tumor treating fields to a subject's body comprising: an array of one or more electrode elements configured to be positioned over the subject's body with a front face and a back face; an anisotropic material layer located on the front face of the array, the anisotropic material layer comprising a front face and a back face, and a non-adhesive flexible layer coupled to the front face of the anisotropic material layer and configured to contact the subject's body optionally via a first adhesive layer; wherein: the anisotropic material layer has a perimeter having at least one concave edge forming at least a portion of a boundary of the array defining an opening between two opposing portions of the anisotropic material layer; the anisotropic material layer has a substantially C-shaped, U-shaped, rounded V-shaped, or annular shaped surface; and the transducer apparatus has a substantially similar shape.

### CROSS REFERENCE TO RELATED APPLICATIONS

This application claims the benefit of and priority to U.S. patent application Ser. No. 18/080,306, filed Dec. 13, 2022, and U.S. Provisional Patent Application No. 63/289,523, filed Dec. 14, 2021, each of which are hereby incorporated by reference in their entirety.

### BACKGROUND

Tumor treating fields (TTFields) are low intensity alternating electric fields within the intermediate frequency range, which may be used to treat tumors as described in U.S. Pat. No. 7,565,205. TTFields are induced non-invasively into the region of interest by electrode assemblies (also called transducer arrays, or, simply, transducers) placed on the patient's body and applying AC voltages between the transducers. Conventionally, transducers used to generate TTFields include a plurality of ceramic disks. One side of each ceramic disk is positioned against the patient's skin, and the other side of each disc has a conductive backing. Electrical signals are applied to this conductive backing, and these signals are capacitively coupled into the patient's body through the ceramic discs. Conventional transducer designs include rectangular arrays of ceramic disks aligned with each other in straight rows and columns (e.g., in a three-by-three arrangement) and attached to the subject's body via a conductive hydrogel.

### SUMMARY OF THE INVENTION

According to some embodiments, a transducer apparatus for delivering tumor treating fields to a subject's body may include an array of one or more electrode elements, the array configured to be positioned over the subject's body with a front face of the array facing the subject's body and a back face facing away from the subject's body; an anisotropic material layer electrically coupled to the array of one or more electrode elements and located on a front side of the front face of the array, the anisotropic material layer comprising a front face and a back face, the back face facing the front face of the array; and a non-adhesive flexible layer having a front face and a back face, the flexible layer coupled to at least a portion of the front face of the anisotropic material layer and configured to contact the subject's body optionally via a first adhesive layer disposed on the front face of the flexible layer; wherein, when viewed from a direction perpendicular to the front face of the anisotropic material layer: the anisotropic material layer has a perimeter having at least one concave edge forming at least a portion of a boundary of the array; the at least one concave edge defines an opening between two opposing portions of the anisotropic material layer; no electrode elements are present in the opening; the anisotropic material layer has a substantially C-shaped, U-shaped, rounded V-shaped, or annular shaped surface; and the transducer apparatus has a shape that is the same shape or substantially same shape as the anisotropic material layer.

According to some embodiments, an assembly for applying tumor treating fields to a subject's body while avoiding at least one area with an anatomic feature or device may include a first transducer array comprising: at least one electrode element; and a first anisotropic material layer comprising a first surface and electrically coupled to the at least one electrode element; wherein when viewed from a direction perpendicular to the first surface: the first surface is substantially C-shaped, U-shaped, rounded V-shaped, or annular shaped wherein: when C-shaped, U-shaped, or rounded V-shaped, two opposing portions of the first surface are spaced apart from each other and connected to each other by a concave edge of the first surface; and when annular shaped, a concave edge defines an opening bounded on all sides by the first surface; and the first transducer array has a shape that is the same shape or substantially same shape as the first surface; and a second transducer array comprising at least one electrode element and a second anisotropic material layer, the second transducer array having a second surface comprising the same or mirror image shape, or a different shape, than the first surface, and the second transducer array has a shape that is the same shape or substantially same shape as the second surface.

According to some embodiments, a transducer apparatus for delivering tumor treating fields to a subject's body may include an array of one or more electrode elements, the array configured to be positioned over the subject's body with a front face of the array facing the subject's body and a back face facing away from the subject's body; an anisotropic material layer electrically coupled to the array and located on a front side of the front face of the array of one or more electrode elements, the anisotropic material layer comprising a front face and a back face, the back face facing the front face of the array; and a non-adhesive flexible layer coupled to at least a portion of the front face of the anisotropic material layer and configured to contact the subject's body optionally via an adhesive layer disposed on the front face of the flexible layer; wherein, when viewed from a direction perpendicular to the front face of the anisotropic material layer: the anisotropic material layer has a perimeter having at least one concave edge forming at least a portion of a boundary of the array; the at least one concave edge defines an opening between two opposing portions of the anisotropic material layer; no electrode elements are present in the opening; the anisotropic material layer has a substantially rounded V-shape comprising a first leg, a second leg, and a vertex portion connecting the first leg to the second leg, and the transducer apparatus has a shape that is the same shape or substantially same shape as the anisotropic material layer.

The above aspect of the invention is exemplary, and other aspects and variations of the invention will be apparent from the following detailed description of embodiments.

### BRIEF DESCRIPTION OF THE DRAWINGS

FIGS. 1A and 1B depict an example of transducers located on a subject's torso according to some embodiments.

FIGS. 2A and 2B depict another example of transducers located on a subject's torso according to some embodiments.

FIG. 3 depicts an example of transducers located on a subject's head according to some embodiments.

FIGS. 4A to 4G depict example layouts of an array of electrode elements on a transducer apparatus according to some embodiments.

FIGS. 5A and 5B depict bottom plan views and top plan views, respectively, of an example transducer apparatus according to some embodiments. FIG. 5C depicts an anisotropic material layer of the example transducer apparatus of FIGS. 5A and 5B in perspective view.

FIG. 6 depicts an exploded view of the example transducer apparatus of FIGS. 5A, 5B, and 5C according to some embodiments.

FIGS. 7A and 7B are cross-sectional views of examples of the structure of various transducer according to some embodiments.

FIG. 8 depicts an example of a configuration of one pair of transducers according to some embodiments.

FIG. 9 depicts an example of an apparatus to determine locations and shapes of transducers on a subject's body for applying TTFields according to some embodiments.

FIG. 10 is a flowchart depicting an example of determining locations and shapes of transducers on a subject's body for applying TTFields according to some embodiments.

### DESCRIPTION OF EMBODIMENTS

As used herein, a substrate having “a substantially C-shaped, U-shaped, rounded V-shaped, or annular shaped surface” means a substrate having “a substantially C-shaped, a substantially U-shaped, a substantially rounded V-shaped, or a substantially annular shaped surface”. In each case, the term “substantially” includes shapes generally recognized as and functionally equivalent to the described shape. For example, the term “substantially U-shaped” includes a rounded flat-bottomed U-shape, and the term “substantially C-shaped” includes a rounded straight-edged C-shape. The two opposing sides, or arms, of the U-shape need not be perfectly parallel and need not be straight. The annular shapes of a substrate may include circular, oval, ovaloid, ovoid, elliptical, etc., for which at least one concave edge defines an interior opening bounded on all sides by the substrate, and wherein the shapes of the interior opening may include circular, oval, ovaloid, ovoid, elliptical, etc. shapes. Further, as described herein, the annular shapes of a substrate, such as circular, oval, ovaloid, ovoid, elliptical, etc., (for which at least one concave edge defines an interior opening bounded on all sides by the substrate) may include a slit (from an exterior boundary of the substrate to the interior opening) which may allow at least temporary access to the interior opening.

This application describes exemplary transducer apparatuses used to apply TTFields to a subject's body for treating one or more cancers. This application also describes exemplary methods to determine the shape and placement of transducer arrays on a subject's body for applying TTFields to the subject's body.

Transducers used to apply TTFields to a subject's body often include multiple electrode elements coupled together on a substrate and attached to the subject's body at a desired location, for example, via an adhesive backing on the skin-facing side of the substrate or a separately applied adhesive. Conventional transducers have relatively large, rectangular, planar surfaces so as to maximize a number of ceramic electrode elements that are located on the transducer and used to apply TTFields to the subject's body. However, the flat rectangular design limits the flexibility of the transducer for contouring to many parts of the subject's body. In addition, if areas on the subject's body are to be avoided by the electrode elements, practitioners have had to place the transducer at a suboptimal location of the subject's body for applying the desired TTFields.

The inventors have now recognized that a need exists for transducers with greater flexibility for comfortably contouring to a subject's body. In addition, the inventors have now recognized that a need exists for transducers that can easily fit around certain anatomical features, chemotherapy ports, and/or other areas to be avoided on the subject's body.

Transducers that are able to effectively contour to a subject's body while avoiding areas that need to remain uncovered and/or would otherwise cause discomfort can be placed at an optimal location on the subject's body. As a result, the transducers can induce TTFields through the subject's body at an ideal location and power level for targeting a region of interest (e.g., tumor) in the subject's body, thereby improving patient outcomes.

The disclosed transducer apparatuses have a substantially C-shaped, U-shaped, rounded V-shaped, or annular shaped surface. The disclosed transducer apparatuses may be readily contoured to the anatomical shape of the subject's body without needing to be reconfigured or placed in a sub-optimal location. The shapes of the disclosed transducer apparatuses provide greater flexibility and continuity for transducer placement around areas such as a breast, ear, chemotherapy port, surgical scar, skin lesion, or any other shaped area on the subject's body that is difficult for the placement of transducers without causing discomfort. In addition, the uniquely shaped transducer apparatuses may be pre-selected by transducer placement planning software in accordance with the disclosed methods. For example, the particular location and shape of the transducers may be chosen together, thereby providing an improved transducer layout to apply a maximum amount of TTFields to the region of interest in the subject's body.

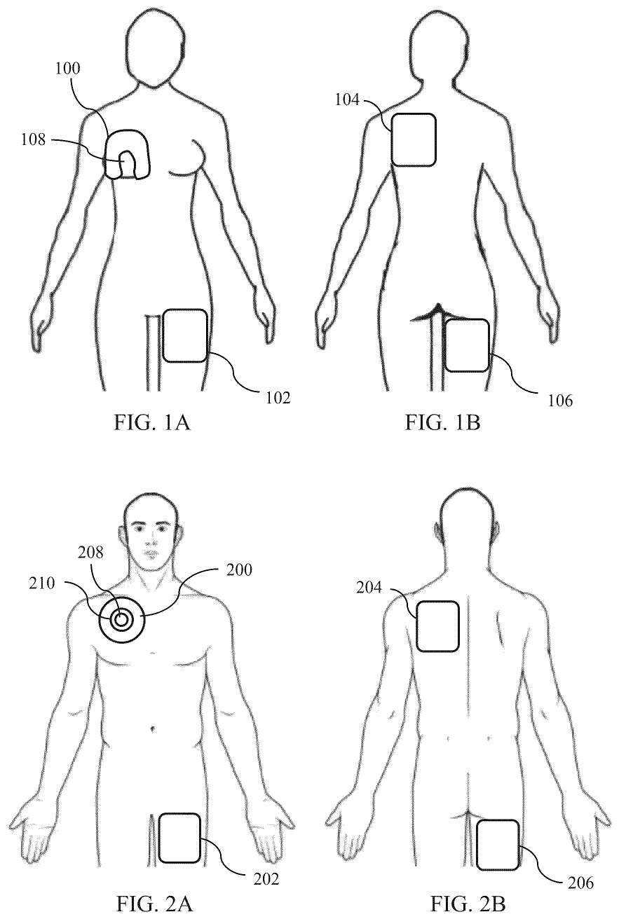

FIGS. 1A and 1B depict an example of transducers positioned at locations on a subject's torso. FIG. 1A depicts a first transducer 100 located on the front of the subject's right breast and a second transducer 102 located on the front of the subject's left thigh. FIG. 1B depicts a third transducer 104 located on the left side of the subject's upper back and a fourth transducer 106 located on the back of the subject's right thigh. The transducers 100, 102, 104, and 106 are transducer arrays each including one or more electrode elements located on a surface that is flexible for contouring to the subject's body.

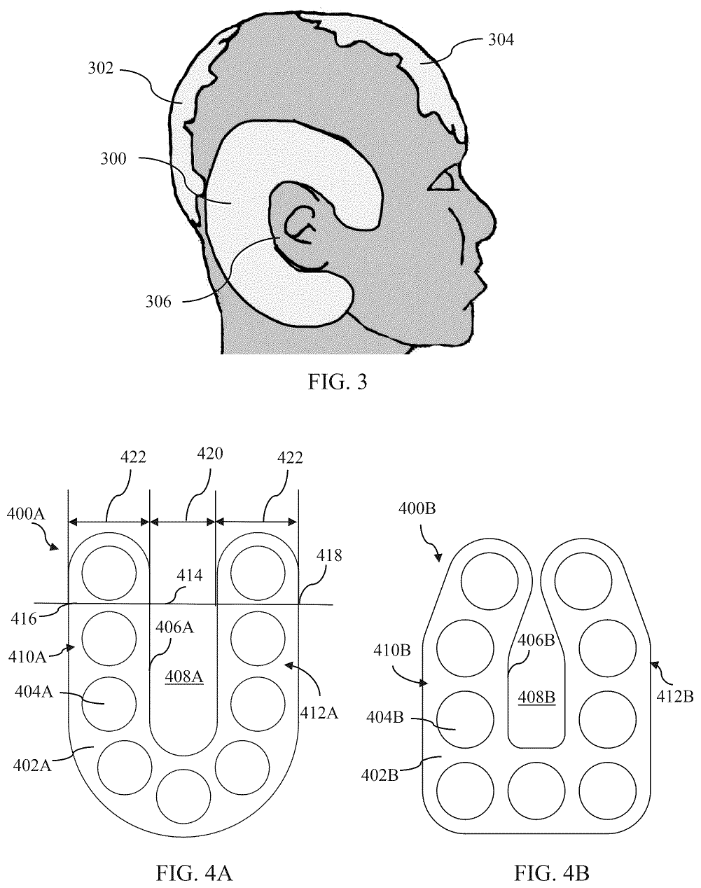

Similarly, FIGS. 2A and 2B depict another example of transducers positioned at locations on a subject's torso. FIG. 2A depicts a first transducer 200 located on the front of the subject's right thorax and a second transducer 202 located on the front of the subject's left thigh. FIG. 2B depicts a third transducer 204 located on the left side of the subject's upper back and a fourth transducer 206 located on the back of the subject's right thigh. The transducers 200, 202, 204, and 206 are transducer arrays each including one or more electrode elements located on a surface that is flexible for contouring to the subject's body.

Transducers arranged on a subject's torso (as shown in FIGS. 1A-2B) are capable of applying TTFields to a tumor in the subject's thorax or abdomen. The transducers may be located at various other combinations of locations on the subject's torso than those of FIGS. 1A-2B.

Both FIGS. 1A/1B and FIGS. 2A/2B illustrate an assembly for applying TTFields to a subject's body while avoiding at least one area with an anatomic feature or device. For example, in FIG. 1A, the surface of the transducer 100 is substantially U-shaped for contouring over a breast of the subject's body while avoiding a nipple 108 of the subject's body. In another example, the surface of the transducer 100 may be substantially C-shaped, rounded V-shaped, or annular shaped for contouring over the breast while avoiding the nipple 108 of the subject's body.

As another example, in FIG. 2A, the surface of the transducer 200 is shaped to avoid a chemotherapy port 208 on the subject's body. In particular, the surface of the transducer 200 is substantially annular shaped and is adapted to be positioned on the subject's body such that an interior opening 210 of the transducer 200 coincides with a location on the subject's body having the chemotherapy port 208. In another example, the surface of the transducer 200 may be substantially C-shaped, U-shaped, or rounded V-shaped and adapted to be positioned on the subject's body with two opposing portions of the transducer surface spaced apart to straddle a location on the subject's body having the chemotherapy port 208. In both examples, no electrode elements of the transducer 200 are located over the chemotherapy port 208. Chemotherapy ports 208 are often inserted into a subject's body prior to the subject receiving TTFields treatment. The transducers disclosed herein may enable the application of TTFields to a region of interest in the subject's thorax or abdomen without interfering with or being affected by the subject's chemotherapy port 208.

Turning back to FIGS. 1A and 1B, one or more other transducers 102, 104, and 106 may have a different shape than the transducer 100. As illustrated, for example, each of the second, third, and fourth transducers 102, 104, and 106 of the assembly has a different shape than the first transducer 100. In some embodiments, each of the second, third, and fourth transducers 102, 104, and 106 may have the same or a substantially similar shape to one or more of the other transducers. As illustrated, the surface of at least one of the transducers 102, 104, and 106 may have a substantially convex shape. More particularly, the surface of at least one of the transducers 102, 104, and 106 may have a rectangular, substantially rectangular with rounded corners (as illustrated), circular, oval, ovaloid, ovoid, or elliptical shape. The transducers 202, 204, and 206 illustrated in FIGS. 2A and 2B have similar shapes as the transducers 102, 104, and 106 shown in FIGS. 1A and 1B, and may also vary as described above for transducers 102, 104, and 106.

In other embodiments, one or more of the other transducers 102, 104, and 106 may have a surface having the same shape or a mirror image shape of the transducer 100 of FIGS. 1A and 1B. For example, two transducers may have the same substantially U-shape for contouring over both breasts of the subject's body while avoiding both nipples. Similarly, one or more of the other transducers 202, 204, and 206 may have a surface having the same shape or a mirror image shape compared to the transducer 200 of FIGS. 2A and 2B.

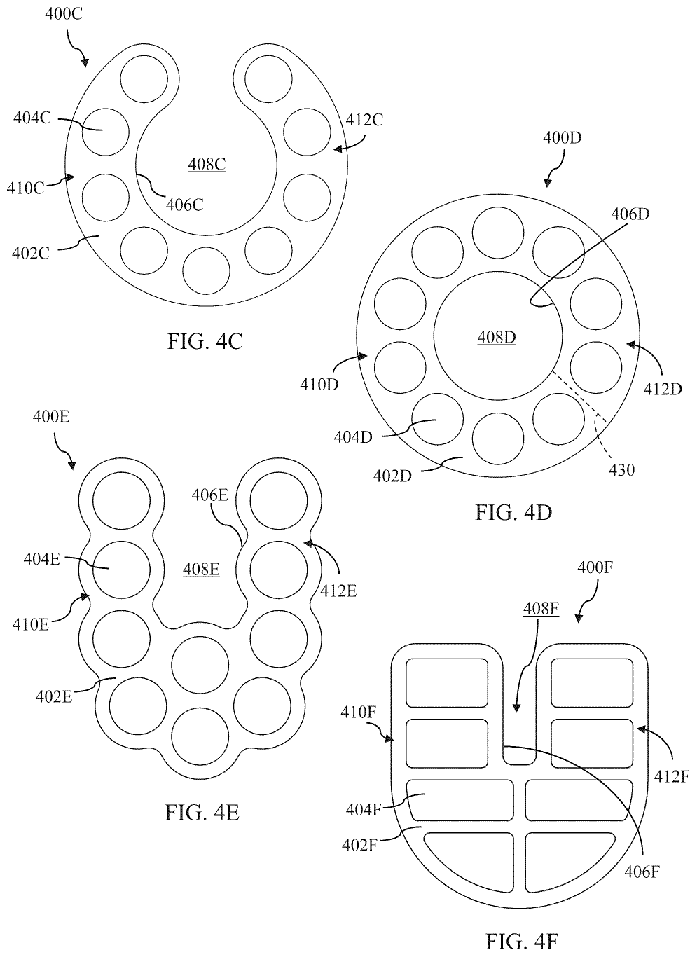

FIG. 3 depicts an example of transducers positioned on the head of a subject's body. FIG. 3 depicts one example of a subject's head on which transducers 300, 302, and 304 are placed in various positions and/or orientations. The transducers 300, 302, and 304 are transducer arrays each including one or more electrode elements located on a surface that is flexible for contouring to the subject's body. Although not shown in the present view of the subject's head, a fourth transducer may be located on an opposite side of the subject's head from the first transducer 300. Transducers arranged on a subject's head (as shown in FIG. 3) are capable of applying TTFields to a tumor in a region of the subject's brain. The transducers may be located at various other combinations of locations on the subject's head than those of FIG. 3.

FIG. 3 illustrates an assembly for applying TTFields to a subject's body while avoiding at least one area with an anatomic feature or device. For example, in FIG. 3, the surface of the transducer 300 is substantially C-shaped for contouring over the subject's head while avoiding an ear 306 of the subject's body. In another example, the surface of the transducer 300 may be substantially U-shaped, rounded V-shaped, or annular shaped for contouring over the subject's head while avoiding the ear 306 of the subject's body.

One or more other transducers 302 and 304 may have a different shape than the transducer 300. As illustrated, for example, each of the second and third transducers 302 and 304 (or a fourth transducer, located opposite the first transducer, not shown) of the assembly may have a different shape than the first transducer 300. In some embodiments, each of the second (302), third (304), and fourth (not shown) transducers may have the same or a substantially similar shape to each other. The surface of at least one of the transducers 302 and 304 may have a substantially convex shape. More particularly, the surface of at least one of the transducers 302 and 304 may have a rectangular, substantially rectangular with rounded corners, circular, oval, ovaloid, ovoid, or elliptical shape. As illustrated, the surface of the at least one of the transducers 302 and 304 may have a scalloped edge.

In other embodiments, one or more of the transducers 302, 304, or a fourth transducer (not shown) may have a surface having the same shape or a mirror image shape of the transducer 300 of FIG. 3. For example, a fourth transducer located opposite the first transducer 300 may have a mirror image of the C-shape of the transducer 300 for contouring over the subject's head while avoiding the other ear of the subject's body.

As disclosed herein, various shapes of transducers may be used to avoid at least one area of the subject's body. The transducers may include, for example, a substantially C-shaped, a substantially U-shaped, a substantially rounded V-shaped, or a substantially annular shaped transducer. In addition, the transducers may be located on the subject's body relative to one or more areas to avoid on the subject's body including, for example, a breast of the subject's body, a nipple of the subject's body, an ear of the subject's body, an eye of the subject's body, a surgical scar or skin lesion of the subject's body, an armpit of the subject's body, or a chemotherapy port disposed in the subject's body.

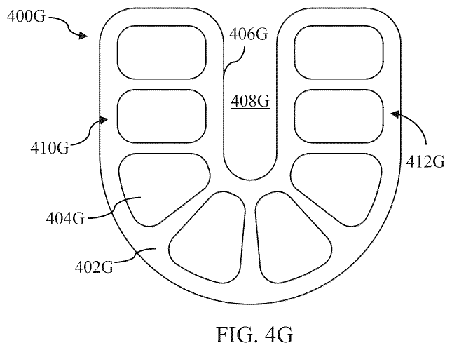

FIGS. 4A-4G depict example transducer apparatuses in accordance with the present disclosure. In each of FIGS. 4A-4G, the transducer apparatus (e.g., 400A, 400B, 400C, 400D, 400E, 400F, and 400G) includes a substrate (e.g., 402A, 402B, 402C, 402D, 402E, 402F, 402G) and an array of electrode elements (e.g., 404A, 404B, 404C, 404D, 404E, 404F, 404G) on the substrate (402A-G). The array of electrode elements (404A-G) is configured to be positioned over the subject's body with a face of the array facing the subject's body. FIGS. 4A-4G each illustrate a transducer apparatus (400A-G) as viewed from a direction perpendicular to this face of the array of electrode elements (404A-G). For all embodiments, the electrode elements may be of any shape; and for any given embodiment, the electrode elements may all be of the same shape, or one or more electrode elements may differ in size and/or shape from other electrode elements. In each of FIGS. 4A-4G, the substrate (402A-G) has at least one concave edge (e.g., 406A, 406B, 406C, 406D, 406E, 406F, 406G) forming at least a portion of a boundary of the array. The at least one concave edge (406A-G) may define an opening (e.g., 408A, 408B, 408C, 408D, 408E, 408F, 408G) between two opposing sides (e.g., 410A/412A, 410B/412B, 410C/412C, 410D/412D, 410E/412E, 410F/412F, 410G/412G) of the substrate (402A-G). No electrode elements (404A-G) are present in the opening (408A-G) in FIGS. 4A-4G. In addition, there may be no hydrogel or adhesive present in the opening (408A-G). The transducers 400A-G may be placed on a subject's body such that the opening (408A-G) is positioned over an area to avoid on the subject's body.

Although the substrate shapes are illustrated in the Figures herein using perfect curves and straight lines, it is to be understood that the terms substantially C-shaped, substantially U-shaped, substantially rounded V-shaped, and substantially annular shaped include all such general shapes; and the substrate shapes may be formed using irregular curves, curves formed from a series of straight lines or arcs of differing radius of curvature, scalloped edges (for example, tracing the outline of one or more individual electrode element shapes), or non-parallel edges. Furthermore, the ends of the two arms of a substrate outline (for example, the arms of a U-shape, rounded V-shape, or C-shape, etc.) that define an opening may be rounded (curved, as shown, for example, in FIG. 4A, FIG. 4B, FIG. 4C and FIG. 4E) or may be straight (flattened curves, as shown, for example, in FIG. 4F and FIG. 4G).

FIG. 4A illustrates a substrate 402A having a substantially U-shaped surface. For a perfect U-shape, the inner concave boundary has a perfectly curved arc and represents exactly one half of a circle before each inner edge boundary of the two arms straightens to form the U-shape (each arm deviating from the parallel position by 0 degrees). The U-shape may incorporate any radius of curvature and therefore the U-shape may be of any size. In some embodiments, the two opposing sides 410A and 412A of the substrate 402A may be substantially straight and extend in substantially parallel directions (e.g., each arm deviating from the parallel position by 2 degrees or less). In some embodiments, the two opposing sides 410A and 412A of the substrate 402A may be substantially straight and extend in substantially parallel directions (e.g., each arm deviating from the parallel position by 20 degrees or less). In such an embodiment, the two arms may align slightly toward each other or align slightly away from each other. In embodiments where the arms are aligned toward each other (not shown), they may not overlap, although they may almost come together, leaving a slight opening. Particularly when the substrate is formed from a flexible material, the latter configuration may allow affixing the transducer apparatus to the body while maneuvering and positioning around a chemotherapy port which is already in place on the body. In embodiments where the two arms are aligned away from each other (not shown), this shape may also be viewed as a rounded V-shape. In some embodiments, the two opposing sides 410A and 412A of the rounded V-shape substrate 402A may be substantially straight and extend away from each other (e.g., each arm deviating from the parallel position by less than 60 degrees, or by less than 45 degrees, but also by more than or equal to 10 degrees, or by more than or equal to 20 degrees). FIG. 4A also illustrates a spatial arrangement between the opening 408A and opposing sides 410A/412A. Specifically, a straight line 414 is drawn through the two opposing sides 410A and 412A of the substrate 402A, from an outer substrate boundary 416 to an opposing outer substrate boundary 418, and across the opening 408A. A distance 420 along the line 414 across the opening 408A may be at least 15% or at least 25% of a distance 422 along the line 414 across one of the opposing sides 410A and 412A of the substrate 402A. In some embodiments, the distance 420 across the opening 408A may be at least 25% or at least 50% of a distance 422 across one of the opposing sides 410A and 412A. In some embodiments, the distance 420 across the opening 408A may be 100% or more of a distance 422 across one of the opposing sides 410A and 412A. This spatial arrangement between the opening (408) and the opposing sides (410/412) may be similar among the transducer apparatuses of FIGS. 4B-4G as well. In each case, the distance 420 along the line 414 is taken at the widest part across the opening 408.

FIG. 4B illustrates a substantially U-shaped substrate 402B having a flat-bottomed U-shaped surface, wherein the two opposing sides (arms) 410B and 412B of the substrate 402B are aligned slightly toward each other. In another embodiment, the substantially U-shaped substrate 402B has a flat-bottomed U-shaped surface, wherein the two opposing sides (arms) 410B and 412B of the substrate 402B are aligned parallel to each other. In another embodiment, the substantially U-shaped substrate 402B has a flat-bottomed U-shaped surface, wherein the two opposing sides (arms) 410B and 412B of the substrate 402B are aligned slightly away from each other as discussed above for the rounded U-shaped embodiments.

FIG. 4C illustrates a substrate 402C having a substantially C-shaped surface. That is, the two opposing sides 410C and 412C of the substrate 402C may be curved. The extent of the C-shape may range from a half-circle, up to the point in which the two arms almost join, leaving a very small opening. As discussed above, the two ends of the C-shape may be rounded or may be straight edged. At the extreme of almost joining and having straight edges at the two ends, this provides a gap or a slit in the transducer which may allow affixing the transducer apparatus to the body while maneuvering and positioning around a chemotherapy port which is already in place on the body.

FIG. 4D illustrates a substrate 402D having a substantially annular shaped surface. That is, the at least one concave edge 406D defines an interior opening 408D bounded on all sides by the substrate 402D. The substrate 402D may have a substantially circular, oval, ovaloid, ovoid, or elliptical shaped surface with the interior opening 408D formed therein. In some embodiments, the interior opening 408D (shown circular in FIG. 4D) may have a substantially circular, oval, ovaloid, ovoid, or elliptical shape to match that of the shape of the substrate. FIG. 4D illustrates an embodiment having a circular shaped surface with the interior opening 408D formed therein. This donut shaped array (circular) may also be fitted over a chemotherapy port which is already in place on the body. Allowing a narrow slit 430 (shown as a dashed line in FIG. 4D) (from an exterior boundary of the substrate to the interior opening 408) may provide a temporary opening in the circular shaped surface and may allow at least temporary access to the interior opening 408 and may facilitate affixing the transducer apparatus to the body while maneuvering and positioning around the chemotherapy port, while also improving flexibility. In some embodiments, the narrow slit 430 may be a score in the transducer apparatus 400D or may include a spacing between two arms of the transducer apparatus 400D. Oval, ovaloid, ovoid, or elliptical shaped transducers may function similarly. For example, an oval, ovaloid, ovoid, or elliptical shape transducer may provide a more appropriate opening to avoid covering a surgical scar or skin lesion.

FIG. 4E illustrates another substrate 402E having an irregular shaped surface. As illustrated in FIG. 4E, the irregular shaped surface of the substrate 402D is substantially U-shaped, and the substrate 402E has a scalloped edge. A scalloped edge may be used with any other transducer shapes (e.g., C-shaped, rounded V-shaped, or annular shaped) disclosed herein. An irregular shaped surface may also be used with any electrode element shape present on the substrate. For example, the irregular shaped surface of the substrate 402D may match (or more nearly match) the perimeter (or expanded version of the perimeter) resulting from tracing the outline of one or more individual electrode element shapes.

FIGS. 4F and 4G illustrate further embodiments of substrates 402F and 402G having a substantially U-shaped surface. As illustrated in both FIG. 4F and FIG. 4G, the ends of the two opposing sides (arms) of the U-shaped substrate that define the opening may be straight (flattened curves). The arms of the U-shape may be of any width and/or length, and, indeed, the two arms of a given substrate need not be identical. Further, as illustrated, for any embodiment, the electrode elements may be of any size, shape and number, and may be the same or different on the substrate. For simplicity, the electrode elements in FIGS. 4A-4E were shown as circular electrode elements. In particular, the shape of the electrode elements for a given shape of substrate may be selected based on availability of electrode elements or may be based on a desire to optimize the filling of the surface shape of the substrate. For example, the rounded triangular electrode elements shown in the U-shaped substrate of FIG. 4G could be used as the only electrode element shape utilized in the circular shaped substrate illustrated in FIG. 4D to provide a higher density of electrode element surface area on the surface of the circular substrate.

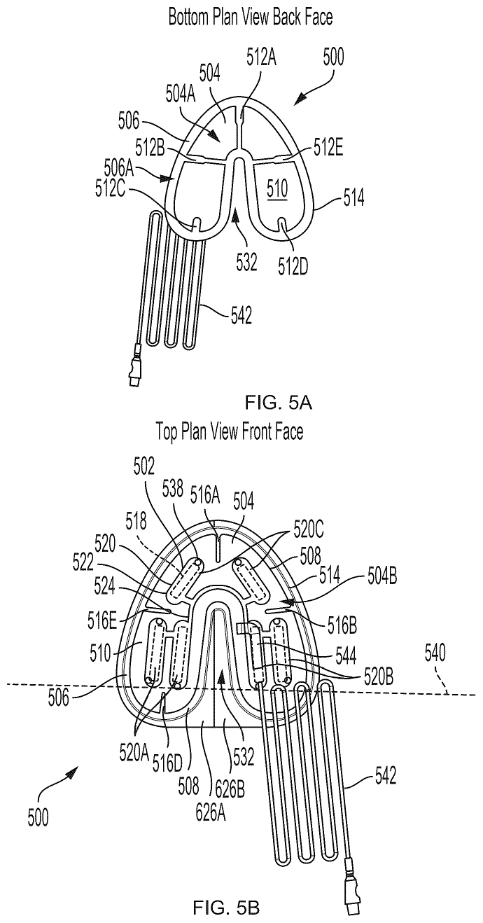

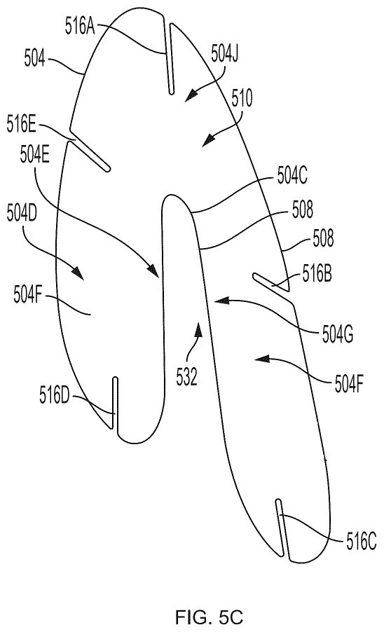

FIGS. 5A and 5B depict bottom plan views (i.e., viewing the skin-facing side) and top plan views (opposite view), respectively, of an example transducer apparatus 500 according to some embodiments. FIG. 5C depicts an anisotropic material layer 504 of the example transducer apparatus 500 of FIGS. 5A and 5B in perspective view. FIG. 6 depicts an exploded view of the example transducer apparatus 500 of FIGS. 5A, 5B, and 5C, but without a transducer wire 542, according to some embodiments.

Referring to FIGS. 5A-C and 6, the transducer apparatus 500 may include an array 502 of one or more electrode elements and an anisotropic material layer 504 coupled to the array 502. The array 502 may have a front face 502A configured to face the subject and a back face 502B opposite the front face configured to face away from the subject. Similarly, the anisotropic material layer 504 may have a front face 504A configured to face the subject and a back face 504B opposite the front face configured to face away from the subject. The anisotropic material layer 504 may be located on a front side of the front face 502A of the array 502. The back face 504B of the anisotropic material layer 504 may face the array 502.

The array 502 may include one or more electrode elements 518 (shown in dashed line in FIG. 5B) connected with a connector of an electrically conductive material (e.g. a flexible printed circuit board (PCB)), for example, and as shown in FIG. 5B, a PCB connector 520. In FIG. 5B, the electrode elements 518 are shown in dashed outline, as the electrode elements 518 are situated between the PCB connector 520 and the anisotropic material layer 504. In FIG. 5B, the transducer apparatus 500 is shown with six electrode elements 518. However, the transducer apparatus 500 may include one, two, three, four, five, six, seven, eight, nine, ten, or more electrode elements. In some embodiments, the array 502 may also include one or more thermistors 538 disposed on its back face 502B.

The connector PCB 520 may include pad portions 522 and line portions 524. The pad portions 522 may be the same size or slightly larger than the electrode elements 518 and may have the same shape or similar shape as the electrode elements 518. In FIG. 5B, the PCB connector 520 includes six pad portions 522. Depending on a desired layout, the PCB connector 520 may include more or less than six pad portions 522. The line portions 524 may connect the pad portions 522. In FIG. 5B, the PCB connector 520 includes three line portions 524. For example, a first line portion 524 may connect the two pad portions 522 of a first leg portion 520A, and a second line portion 524 may connect the two pad portions 522 of a second leg portion 520B. Further, a third line portion 524 may connect the two pad portions 522 of a vertex portion 520C, the right pad portion 522 of the first leg portion 520A, and the left leg portion of the second leg portion 520B. Depending on a desired layout, the PCB connector 520 may include more or less than three line portions 524, and the line portions 524 may connect the pad portions 522 in various combinations.

The PCB connector 520 may be of a similar or substantially similar outline or shape relative to the anisotropic material layer 504. As depicted in FIGS. 5B and 5C, the PCB connector 520 includes the first leg portion 520A disposed on a first leg 504D of the anisotropic material layer 504, the second leg portion 520B disposed on a second leg 504F of the anisotropic material layer 504, and the vertex portion 520C disposed on a vertex portion 504J of the anisotropic material layer 504. In such an embodiment, each of the first leg portion 520A, second leg portion 520B, and vertex portion 520C may include at least one electrode element 518 disposed thereon.

The anisotropic material layer 504 may be any conductive layer having different thermal and/or electrical conductivities in a direction perpendicular to the front face 504A of the anisotropic material layer 504 than in directions that are parallel to the front face 504A. The anisotropic material layer 504 may be anisotropic with respect to electrical conductivity properties, anisotropic with respect to thermal conductivity properties, or both. This allows the anisotropic material layer 504 to spread out current and/or heat over a larger surface area. In each case, this lowers the temperature of hot spots and raises the temperature of cooler regions when a given AC voltage is applied to the array 502. Accordingly, the current can be increased without exceeding a safety temperature threshold at any point on the subject's skin. The anisotropic material layer 504 may be a sheet of synthetic graphite. The anisotropic material layer 504 may be a sheet of pyrolytic graphite, graphitized polymer film, a graphite foil made from compressed high purity exfoliated mineral graphite, or some other material. Other details regarding the anisotropic material layer 504 and properties thereof are described in U.S. Patent Application Publication No. 2023/0037806 A1, Wasserman et al., Feb. 9, 2023, which is hereby incorporated herein by reference in its entirety.

The anisotropic material layer 504 may be a rounded V-shape or a boomerang shape. Alternatively, the anisotropic material layer 504 may be any other suitable shape including but not limited to a rectangular shape, a rounded rectangular shape, a triangular shape, a rounded triangular shape, a circular shape, an oval shape, an annular shape, a C-shape, U-shape, a substantially rectangular shape, a substantially rounded rectangular shape, a substantially triangular shape, a substantially rounded triangular shape, a substantially circular shape, a substantially oval shape, a substantially C-shape, a substantially U-shape, a substantially rounded V-shape, or a substantially boomerang shape. In some embodiments, the transducer apparatus 500 may be substantially the same shape as the anisotropic material layer 504.

As depicted in FIG. 5C, the anisotropic material layer 504 may have at least one concave edge 504C forming at least a portion of a perimeter 508 of the anisotropic material layer 504. The at least one concave edge 504C may define an opening 532 between two opposing portions or legs 504D, 504F of the anisotropic material layer 504. The anisotropic material layer 504 may include a vertex portion 504J connecting the first portion 504D to the second portion 504F. The transducer apparatus 500 may be configured such that no electrode elements are present in the opening 532. In use, the transducer apparatus 500 may be placed on a subject's body such that the opening 532 is positioned over an area to avoid on the subject's body, such as a nipple, chemotherapy port, or any other body part. In this way, the opposing portions 504D, 504F may be spaced apart by the opening 532 such that the opposing portions 504D, 504F may straddle a nipple, chemotherapy port, or other body part.

In some embodiments, for example those having a C-shaped or substantially C-shaped anisotropic material layer 504 the two opposing portions 504D, 504F may be curved, such as depicted in FIG. 4C. In other embodiments, for example those have a U-shaped, substantially U-shaped, rounded V-shaped, or substantially rounded V-shaped anisotropic material layer 504, the two opposing portions 504D, 504F of the anisotropic material layer 504 may have a first inner side 504E and second inner side 504G (FIG. 5C), respectively. The first inner side 504E may border the opening between the two opposing portions 504D, 504F, and the second inner side 504G may border the opening between the two opposing portions and is opposite the first inner side. The first inner side 504E and second inner side 504G may be substantially straight and extend in substantially parallel directions, such as depicted in FIGS. 4A, 4F, and 4G; alternatively, the sides 504E, 504G may deviate from being parallel by less than 30°, 45°, 60°, or any other suitable angle, such as depicted in FIG. 4B. Furthermore, when a straight line (e.g., straight line 540, FIG. 5B) is drawn through the sides 504E, 504G and across the opening 532, the distance along the line across the opening may be at least 15% or at least 25% of the distance along the line across one of the opposing portions 504D, 504F of the anisotropic material layer 504. In yet another embodiment, for example those having an annular-shaped anisotropic material layer 504, such as depicted in FIG. 4D, the opening 532 defined by the at least one concave edge 504C may be bounded on all sides by the anisotropic material layer 504 such that the anisotropic material layer 504 has a substantially circular, oval, ovaloid, ovoid, or elliptical shaped with the opening 532 formed therein.

As shown in FIGS. 5C and 6, when viewed from the front face 504A of anisotropic material layer 504, anisotropic material layer 504 may include one or more slits 516A-E or other cutout features to improve the flexibility of the transducer apparatus 500. In some embodiments, the slits 516A-E may extend from the perimeter or exterior boundary 508 towards the interior portion 510 of the anisotropic material layer 504. In an embodiment wherein the anisotropic material layer 504 is a substantially circular, oval, ovaloid, ovoid, or elliptical shape (e.g., such as depicted in FIG. 4D), the anisotropic material layer 504 may include a slit running from an exterior boundary 508 of the anisotropic material layer 504 to the opening 532 allowing at least a temporary opening (530, not shown) in the annular shape (such as the slit 430 in FIG. 4D).

The transducer apparatus 500 may further include a non-adhesive flexible layer 506 having a front face 506A and a back face 506B. The flexible layer 506 may be at least partially disposed on the front face 504A of the anisotropic material layer 504 and may further be disposed over at least a portion of an outer perimeter 508 of the anisotropic material layer 504. In this way, the flexible layer 506 may define a seal between the anisotropic material layer 504 and a subject's body. In FIG. 5B, the flexible layer 506 is partially shown as extending beyond the perimeter 508 of the anisotropic material layer 504. In other embodiments, the flexible layer 506 may not extend beyond the perimeter 508 of the anisotropic material layer 504; for example, the flexible layer 506 may extend up to the perimeter 508 of the anisotropic material layer 504 such that the perimeter edge of the flexible layer 506 may be coincident with the perimeter edge 508 of the anisotropic material layer 504. The flexible layer 506 may be any suitable thickness, for example, and without limitation, less than or equal to 0.1 mm, 0.5 mm, 1.0 mm, 1.5 mm, 2 mm, or 5 mm. Furthermore, the flexible layer 506 may include adhesive. In some embodiments, the flexible layer 506 may have a skin-contact adhesive layer (for example, a biocompatible adhesive) disposed on the front face 506A to couple the transducer apparatus 500 to a subject's body.

The flexible layer 506 may be electrically non-conductive. For example, the flexible layer 506 may be made from a compressible material including but not limited to foam, rubber, elastomer, tape, bandage, or plaster. In some embodiments, the flexible layer 506 may be an adhesive coated foam. In some embodiments, the flexible layer 506 may be 3M™ Tegaderm™ Transparent Film Dressing Frame, which may include adhesive on one side facing the patient's body and facing away from the anisotropic material layer 504. Alternatively, the flexible layer 506 may not include such an adhesive so that the transducer apparatus 500 instead includes an adhesive layer such as an electrically conductive skin contact adhesive layer, which may be positioned on the front face of the anisotropic material layer 504. In some embodiments, the flexible layer 506 may be coated with an adhesive, and the anisotropic material layer 504 may be coated with an electrically conductive adhesive layer. In some embodiments, both adhesives of the flexible layer 506 and the anisotropic material layer 504 may be biocompatible adhesives.

In some embodiments, the flexible layer 506 may be coupled to the anisotropic material layer 504, by an adhesive layer disposed on the front face 504A of the anisotropic material layer 504, and this adhesive layer may couple the anisotropic material layer 504 to the flexible layer 506; this adhesive layer may or may not have an adhesive strength which is less than the adhesive strength of the adhesive layer disposed on the front face 506A of the flexible layer 506. In some embodiments, the back face of the flexible layer 506B may also have a layer of adhesive to help adhere the flexible layer 506 to the anisotropic material layer 504. As will be explained in further detail below, the flexible layer 506 may be disposed about a perimeter 508 of the anisotropic material layer 504; in this way, the flexible layer 506 may have a perimeter portion outline shape which is substantially similar to that of the anisotropic material layer 504 and which may partially cover an interior portion 510 of the anisotropic material layer 504. In some embodiments, the perimeter 514 of the flexible layer 506 may extend beyond the perimeter 508 of the anisotropic material layer 504; in other embodiments, the perimeter 514 of the flexible layer 506 may extend up to the same boundary as that of the perimeter 508 of the anisotropic material layer 504. In some embodiments, the flexible layer 506 may cover at least 1% and at most 40% of the surface area of the anisotropic material layer 504 when viewed from its front face 504A.

The flexible layer 506 may include at least one radial portion 512A-E extending from the perimeter 508 of the anisotropic material layer 504 towards an the interior 510 of the anisotropic material layer 504. In some embodiments, the at least one radial portion 512A-E may extend along the length of, and cover, the slits 516A-E, respectively, such that slits 516A-E are not visible when viewed from the front face 504A of anisotropic material layer 504 (as in FIG. 5A). Furthermore, radial portions 512A-E may have a single thickness (in a plane parallel to the face of the array) along their entire length or may have a first portion 636A having a first thickness, a second portion 636B having a second thickness, and so on. The radial portions 512A-E may extend only partially towards the interior portion 510 of the anisotropic material layer 504, or may extend from a first point along the perimeter 508 of the flexible layer 506 to a second point along the perimeter 508. In one example shown in FIG. 5A, a first portion (636A) of the respective radial portions 512A, 512B, and 512E may extend from a first point along the perimeter 508 to an endpoint of the slits 516A, 516B, and 516E, respectively, and a second portion (636B) of the respective radial portions 512A, 512B, and 512E may extend from the endpoint of the slits 516A, 516B, and 516E to a second point along the perimeter 508 such that the first portion 636A has a first thickness and the second portion 636B has a second thickness which is less than the first thickness.

The transducer apparatus 500, 600 may also include an adhesive layer disposed on the front face 504A of the anisotropic material layer 504. In some embodiments, this adhesive layer may have a lower peel strength or tack strength than the adhesive of the front face 506A of the flexible layer 506.

In some embodiments, the transducer apparatus 500 may include a transducer wire 542 and a connector 544 disposed on the array 502 for connecting the transducer apparatus 500 to transducer wire 542. In some embodiments, the transducer apparatus 500 may not initially include the transducer wire 542, which may later be added by the subject or caregiver of the subject.

As shown in FIG. 6, the transducer apparatus 600 may include a substrate 628, which may assist in holding together various components of the transducer apparatus 600. In some embodiments, the transducer apparatus 600 may be affixed to the subject's body with the aid of the substrate 628. Suitable materials for the substrate 628 may include, for example, cloth, foam, flexible plastic, and/or a conductive medical gel or adhesive. In some embodiments, the substrate 628 may take the form of an adhesive bandage (e.g., a medical bandage). In other embodiments, the substrate 628 may not extend beyond the perimeter of the other transducer components and may not be involved in adhering the transducer to the subject's body.

As is also shown in FIGS. 4B and 6, the transducer apparatus 600 may be packaged prior to use with peel-off covers 626A, 626B (collectively 626, release liners) disposed on the front face 506A of the flexible layer 506.

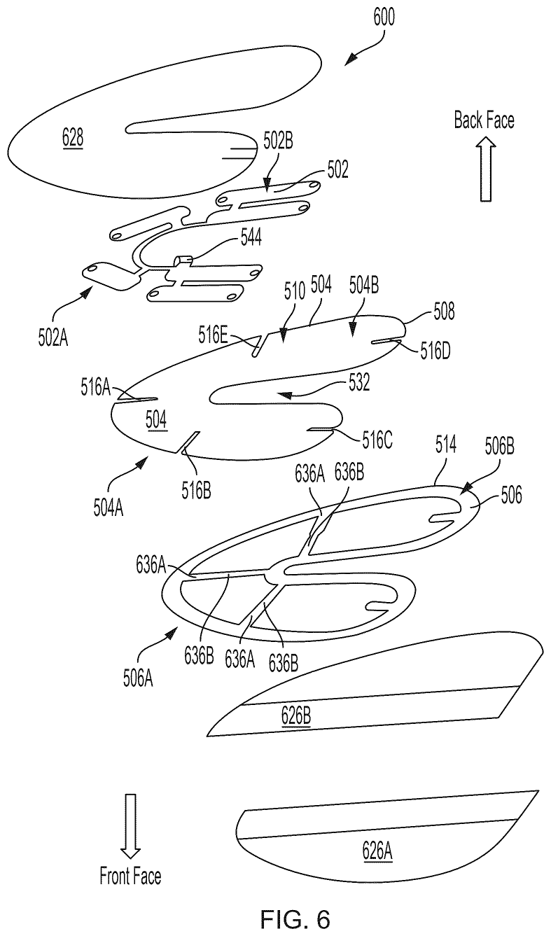

The structure of the transducer apparatuses may take many forms. In FIG. 7A, the transducer apparatus 700A has a plurality of electrode elements 702A positioned on a substrate 704A. The substrate 704A is configured for attaching the transducer 700A to a subject's body. Suitable materials for the substrate 704A include, for example, cloth, foam, flexible plastic, and/or a conductive medical gel. The transducer apparatus 700A may be affixed to the subject's body via the substrate 704A (e.g., via an adhesive backing and/or the conductive medical gel). The transducer may be conductive or non-conductive. FIG. 7B depicts another example of the structure of the transducer apparatus 700B. In this example, the transducer apparatus 700B includes a plurality of electrode elements 702B that are electrically and mechanically connected to one another without a substrate. In one example, electrode elements 702B are connected to each other through conductive wires 706B.

The transducer apparatuses 700A and 700B may comprise arrays of substantially flat electrode elements 702A and 702B, respectively. The array of electrode elements may be capacitively coupled. In one example, the electrode elements 702A and 702B are non-ceramic dielectric materials positioned over a plurality of flat conductors. Examples of non-ceramic dielectric materials positioned over flat conductors include polymer films disposed over pads on a printed circuit board or over flat pieces of metal. In another example, the electrode elements 702A and 702B are ceramic elements. In some embodiments, the dielectric constant of the dielectric material is at least 10; in some embodiments, the dielectric constant of the dielectric material is at least 20.

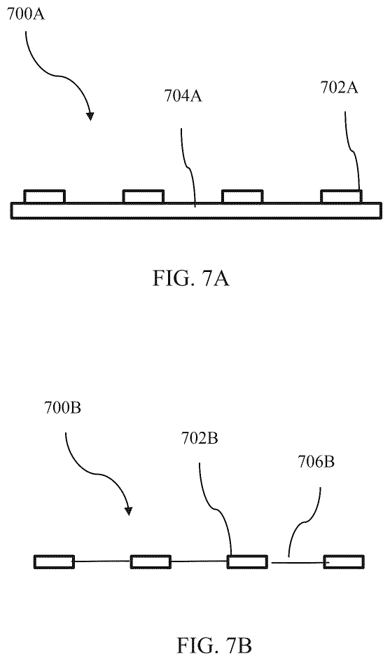

FIG. 8 depicts an example configuration of one pair of transducer apparatuses 802 and 804. In this example, the apparatus 800 comprises transducer apparatuses 802 and 804. The first transducer apparatus 802 includes 13 electrode elements 806 which are positioned on a substrate 808 and electrically and mechanically connected to one another through a conductive wiring 810. Similarly, the second transducer apparatus 804 includes 14 electrode elements 812 which are positioned on a substrate 814 and electrically and mechanically connected to one another through a conductive wiring 816. The substrate 814 may be substantially U-shaped (as shown), C-shaped, rounded V-shaped, or annular shaped. Although the outline of the substrate 814 is depicted with straight lines and sharp vertices in FIG. 8, the outline may be curved as, for example, in FIG. 4A, 4C or 5B. The two substrates 808 and 814 may have the same, mirror image, or different shapes from each other. The transducer apparatuses 802 and 804 may be connected to an AC voltage generator 818 and a controller 820. The AC voltage generator 818 is adapted to be coupled to the transducer apparatuses 802 and 804 and is capable of providing a first voltage to the transducer 802 and a second voltage to the transducer 804. The controller 820 may include one or more processors and memory accessible by the one or more processors. The memory may store instructions that, when executed by the one or more processors, control the AC voltage generator 818 to induce an electric field between the two transducer apparatuses 802 and 804. The AC signal may provide an alternating current waveform at frequencies in a range of from about 50 kHz to about 1 MHz, such as, for example, 100 kHz to 300 kHz; and the field intensity may be from 1-10 V/cm, for example, 1-5 V/cm.

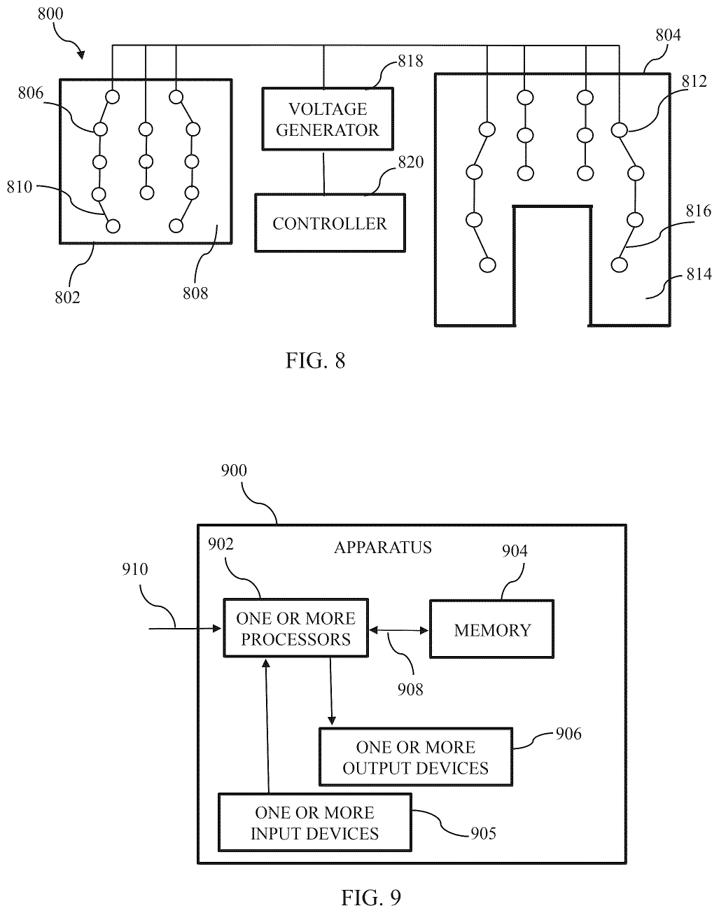

FIG. 9 depicts an exemplary apparatus to determine locations and shapes of transducers for applying TTFields. In this example, the apparatus 900 includes one or more processors 902, a memory 904, one or more input devices 905, and one or more output devices 906. The memory 904 is accessible by the one or more processors 902 via a link 908 so the one or more processors 902 can read information from and write information to the memory 904. The memory 904 stores instructions that, when executed by the one or more processors 902, cause the apparatus 900 to perform one or more methods disclosed herein. The one or more processors 902 may receive inputs 910 (e.g., user inputs of image data) and, based on the inputs 910, make one or more recommendations of transducer shapes and/or locations to a user, which are output by the output devices 906. The inputs 910 may be from a network link or from one or more input devices 905.

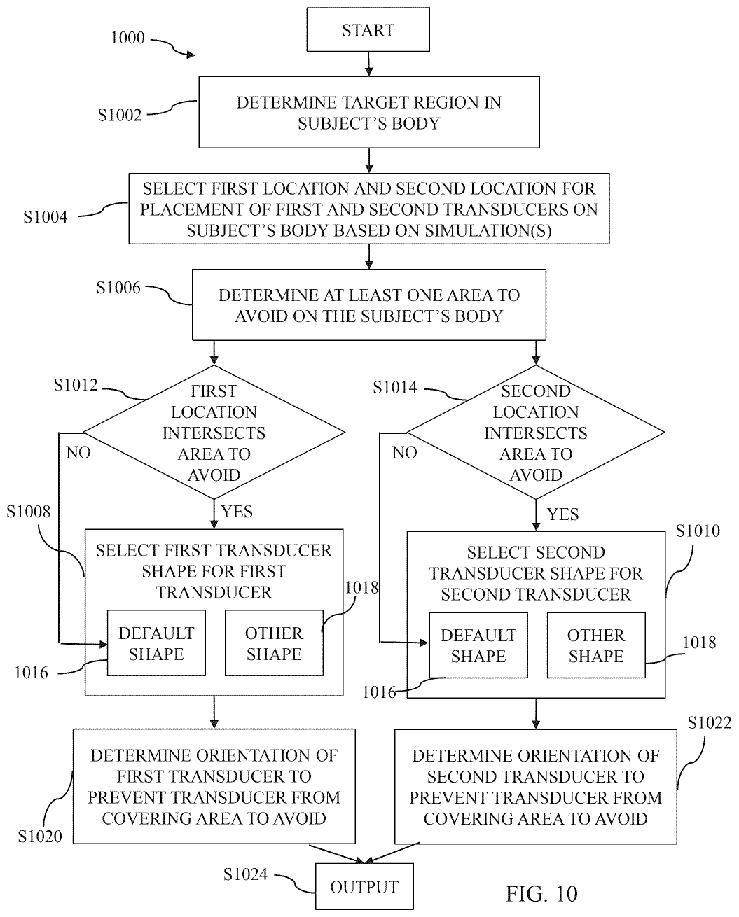

FIG. 10 is a flowchart depicting an example method 1000 of determining the shape and placement of transducers (e.g., transducer apparatuses discussed herein) on a subject's body for applying TTFields. As illustrated, the method 1000 may begin at step S1002 with determining a target region in the subject's body. This may be accomplished by, for example, analyzing one or more sets of image data (e.g., magnetic resonance imaging (MRI) data, computer tomographic (CT) data, etc.) to determine an approximate location and/or 3D volume of the target region in the subject's body.

At step S1004, the method 1000 includes selecting a first location on the subject's body for placement of a first transducer and a second location on the subject's body for placement of a second transducer, based on one or more simulations of an electric field distribution through the target region in the subject's body. This may involve, for example, performing one or more simulations (using a simulation algorithm) of the expected electric field distribution through the target region of the subject's body based on image data associated with the subject's body. More particularly, the determination may be made by comparing simulations for different possible transducer location pairs, and ranking/recommending a pair of transducer locations based on expected electric field distributions through the target region.

At step S1006, the method 1000 includes determining at least one area to avoid for placement of a transducer array on the subject's body. The at least one area to avoid may correspond to an anatomical feature of the subject's body or a device located in the subject's body, as discussed above. The at least one area to avoid may be determined, for example, via an image processing module identifying landmarks (e.g., anatomical features and/or devices) depicted in one or more images included with image data of the subject's body. The image processing module may use one or more object identification and/or tracking algorithms to determine/detect the locations of one or more landmarks. In another example, the at least one area to avoid may be identified based on user inputs including, for example, an indication of the presence and/or approximate location of a chemotherapy port in the subject's body, body measurements (e.g., breast size measurements), and others.

At step S1008, the method 1000 includes selecting a first transducer shape for the first transducer based on the first location and the at least one area to avoid on the subject's body. Similarly, at step S1010, the method 1000 includes selecting a second transducer shape for the second transducer based on the second location and the at least one area to avoid on the subject's body. The second transducer shape may be the same shape as, a mirror image shape of, or a different shape from, the first transducer shape.

Selecting the first transducer shape may involve, for example, determining at step S1012 whether the first location overlaps at least a portion of the at least one area to avoid. Similarly, selecting the second transducer shape may involve determining at step S1014 whether the second location overlaps at least a portion of the at least one area to avoid. Upon determining that either of the first location or second location does not overlap at least a portion of the at least one area to avoid (“NO”), then a default transducer shape 1016 may be selected for the corresponding first transducer (S1008) or second transducer (S1010). The default transducer shape 1016 may be a rectangular, substantially rectangular with rounded corners, circular, oval, ovaloid, ovoid, or elliptical shape. Upon determining that either of the first location or second location overlaps at least a portion of the at least one area to avoid (“YES”), then a shape 1018 other than the default shape 1016 may be selected for the corresponding first transducer (S1008) or second transducer (S1010). The other shape 1018 may include a substantially C-shape, U-shape, rounded V-shape, or annular shape. In some embodiments, the method 1000 may include selecting the particular other shape 1018 based on factors such as, for example, the relative size and position of the overlapping portion of the at least one area to avoid compared to a default shaped transducer located at the same position on the subject's body.

In an example, at step S1020 the method 1000 may further include determining an orientation of the first transducer having the first transducer shape at the first location of the subject's body to prevent the first transducer from covering at least a portion of the at least one area to avoid located at the first location. Similarly, at step S1022, the method 1000 may further include determining an orientation of the second transducer having the second transducer shape at the second location of the subject's body to prevent the second transducer from covering at least a portion of the at least one area to avoid located at the second location.

At step S1024, the method 1000 includes outputting the recommended first transducer shape, first location, second transducer shape, and second location to a user (e.g., via an output on a user interface). Step S1024 may also include outputting a recommended orientation of one or both of the first transducer and the second transducer to the user. The outputs may be in the form of visual notifications for transducer array placement. That is, the one or more recommended placement positions for the one or more transducer arrays and the one or more areas to avoid may be displayed to the user. The notification may visually instruct the user where to place a transducer array to 1) avoid the one or more areas to avoid on the subject's body, and 2) receive an optimized electric field applied to the target region.

### ILLUSTRATIVE EMBODIMENTS

The invention includes other illustrative embodiments, such as the following, and any combination of these illustrative embodiments (or portions thereof) may be made to define an embodiment.

Illustrative Embodiment 1: A transducer apparatus for delivering tumor treating fields to a subject's body, the transducer apparatus comprising: an array of one or more electrode elements, the array configured to be positioned over the subject's body with a front face of the array facing the subject's body and a back face facing way from the subject's body; an anisotropic material layer electrically coupled to the array of one or more electrode elements and located on a front side of the front face of the array, the anisotropic material layer comprising a front face and a back face, the back face facing the front face of the array; and a non-adhesive flexible layer having a front face and a back face, the flexible layer coupled to at least a portion of the front face of the anisotropic material layer and configured to contact the subject's body optionally via a first adhesive layer disposed on the front face of the flexible layer; wherein, when viewed from a direction perpendicular to the front face of the anisotropic material layer: the anisotropic material layer has a perimeter having at least one concave edge forming at least a portion of a boundary of the array; the at least one concave edge defines an opening between two opposing portions of the anisotropic material layer; no electrode elements are present in the opening; the anisotropic material layer has a substantially C-shaped, U-shaped, rounded V-shaped, or annular shaped surface; and the transducer apparatus has a shape that is the same shape or substantially same shape as the anisotropic material layer.

Illustrative Embodiment 2: The transducer apparatus of Illustrative Embodiment 1, wherein when viewed from the direction perpendicular to the front face of the anisotropic material layer: the anisotropic material layer has a substantially C-shaped surface; and the two opposing portions of the anisotropic material layer are curved.

Illustrative Embodiment 3: The transducer apparatus of Illustrative Embodiment 1, wherein when viewed from the direction perpendicular to the front face of the anisotropic material layer: the anisotropic material layer has a substantially U-shaped or rounded V-shaped surface; and the two opposing portions of the anisotropic material layer comprise a first inner side and second inner side, respectively, which are substantially straight and extend in substantially parallel directions, or deviate from being parallel by less than 60°, wherein the first inner side borders the opening between the two opposing portions and the second inner side borders the opening between the two opposing portions and is opposite the first inner side.

Illustrative Embodiment 4: The transducer apparatus of Illustrative Embodiment 1, wherein when viewed from the direction perpendicular to the front face of the anisotropic material layer: the anisotropic material layer has a substantially annular shaped surface; the opening defined by the at least one concave edge is bounded on all sides by the anisotropic material layer; and the anisotropic material layer has a substantially circular, oval, ovaloid, ovoid, or elliptical shaped surface with the opening formed therein.

Illustrative Embodiment 5: The transducer apparatus of Illustrative Embodiment 4, wherein the substantially circular, oval, ovaloid, ovoid, or elliptical shaped surface comprises a slit running from an exterior boundary of the anisotropic material layer to the opening allowing at least a temporary opening in the annular shape.

Illustrative Embodiment 6: The transducer apparatus of Illustrative Embodiment 1, wherein when viewed from the direction perpendicular to the front face of the anisotropic material layer: the first opposing portion defines a first opposing side; the second opposing portion defines a second opposing side; and when a straight line is drawn through the first and second opposing sides of the anisotropic material layer and across the opening, a distance along the line across the opening is at least 15% of a distance along the line across one of the opposing portions of the anisotropic material layer.

Illustrative Embodiment 7: The transducer apparatus of Illustrative Embodiment 1, wherein the front face of the anisotropic material layer comprises a perimeter and an interior portion; the anisotropic material layer comprises at least one slit extending from the perimeter of the front face of the anisotropic material layer towards the interior portion of the front face of the anisotropic material layer; and when viewed from the direction perpendicular to the front face of the anisotropic material layer, a first portion of the flexible layer extends along and covers the at least one slit.

Illustrative Embodiment 8: The transducer apparatus of Illustrative Embodiment 7, wherein when viewed from the direction perpendicular to the front face of the anisotropic material layer, the first portion of the flexible layer further extends from a first point of the perimeter of the front face of the anisotropic material layer to a second point of the perimeter of the front face of the anisotropic material layer.

Illustrative Embodiment 9: The transducer apparatus of Illustrative Embodiment 8, wherein when viewed from the direction perpendicular to the front face of the anisotropic material layer, the first portion of the flexible layer comprises a thicker portion covering the at least one slit and a thinner portion not covering the at least one slit.

Illustrative Embodiment 10: The transducer apparatus of Illustrative Embodiment 7, wherein when viewed from the direction perpendicular to the front face of the anisotropic material layer, the flexible layer further comprises a second portion disposed about a perimeter of the anisotropic material layer.

Illustrative Embodiment 11: The transducer apparatus of Embodiment 1, wherein the anisotropic material layer comprises graphite.

Illustrative Embodiment 11A: The transducer apparatus of Illustrative Embodiment 11, wherein the anisotropic material layer comprises a sheet of synthetic graphite.

Illustrative Embodiment 11B: The transducer apparatus of Illustrative Embodiment 11, wherein the anisotropic material layer comprises a sheet of pyrolytic graphite, graphitized polymer film, or a graphite foil made from compressed high purity exfoliated mineral graphite.

Illustrative Embodiment 11C: The transducer apparatus of Illustrative Embodiment 1, wherein: when viewed from the direction perpendicular to the front face of the anisotropic material layer, the flexible layer comprises a front face facing the subject's body and a back face facing the front face of the anisotropic material layer, and the front face of the flexible layer comprises the first adhesive disposed thereon.

Illustrative Embodiment 12: The transducer apparatus of Illustrative Embodiment 1; wherein the first adhesive has a first adhesive strength; and the front face of the anisotropic material layer comprises a second adhesive having a second adhesive strength less than the first adhesive strength.

Illustrative Embodiment 12A: The transducer apparatus of Illustrative Embodiment 1, further comprising a substrate disposed on the back face of the array of one or more electrode elements, the substrate comprising an adhesive layer on at least one surface of the substrate.

Illustrative Embodiment 13: An assembly for applying tumor treating fields to a subject's body while avoiding at least one area with an anatomic feature or device, the assembly comprising: a first transducer array comprising: at least one electrode element; and a first anisotropic material layer comprising a first surface and electrically coupled to the at least one electrode element; wherein when viewed from a direction perpendicular to the first surface: the first surface is substantially C-shaped, U-shaped, rounded V-shaped, or annular shaped wherein: when C-shaped, U-shaped, or rounded V-shaped, two opposing portions of the first surface are spaced apart from each other and connected to each other by a concave edge of the first surface; and when annular shaped, a concave edge defines an opening bounded on all sides by the first surface; and the first transducer array has a shape that is the same shape or substantially same shape as the first surface; and a second transducer array comprising at least one electrode element and a second anisotropic material layer, the second transducer array having a second surface comprising the same or mirror image shape, or a different shape, than the first surface, and the second transducer array has a shape that is the same shape or substantially same shape as the second surface.

Illustrative Embodiment 14: The assembly of Illustrative Embodiment 13, wherein the first surface of the first anisotropic material layer is substantially C-shaped, U-shaped, or rounded V-shaped and is adapted to be positioned on the subject's body such that the two opposing portions of the first surface are spaced apart to straddle a first location on the subject's body coinciding with a nipple or the first location on the subject's body having a chemotherapy port, and no electrode elements of the first transducer array are located over the nipple or chemotherapy port.

Illustrative Embodiment 15: The assembly of Illustrative Embodiment 13, wherein the first surface of the first anisotropic material layer is substantially annular shaped and is adapted to be positioned on the subject's body such that the opening coincides with a first location on the subject's body coinciding with a nipple or the first location on the subject's body having a chemotherapy port, and no electrode elements of the first transducer array are located over the nipple or chemotherapy port.

Illustrative Embodiment 15A: The transducer apparatus of Embodiment 13 further comprising a flexible layer of foam coupled to at least a portion of the front face of the anisotropic material layer, wherein the flexible layer of foam has adhesive disposed at least on a front face of the foam facing the subject's body.

Illustrative Embodiment 15B: The transducer apparatus of Embodiment 13, wherein the first anisotropic material layer and/or the second anisotropic material layer comprises graphite.

Illustrative Embodiment 15C: The transducer apparatus of Illustrative Embodiment 15B, wherein the first anisotropic material layer and/or the second anisotropic material layer comprises a sheet of synthetic graphite.

Illustrative Embodiment 15B: The transducer apparatus of Illustrative Embodiment 11, wherein the first anisotropic material layer and/or the second anisotropic material layer comprises a sheet of pyrolytic graphite, graphitized polymer film, or a graphite foil made from compressed high purity exfoliated mineral graphite.

Illustrative Embodiment 16: A transducer apparatus for delivering tumor treating fields to a subject's body, the transducer apparatus comprising: an array of one or more electrode elements, the array configured to be positioned over the subject's body with a front face of the array facing the subject's body and a back face facing away from the subject's body; an anisotropic material layer electrically coupled to the array and located on a front side of the front face of the array of one or more electrode elements, the anisotropic material layer comprising a front face and a back face, the back face facing the front face of the array; and a non-adhesive flexible layer coupled to at least a portion of the front face of the anisotropic material layer and configured to contact the subject's body optionally via an adhesive layer disposed on the front face of the flexible layer; wherein, when viewed from a direction perpendicular to the front face of the anisotropic material layer: the anisotropic material layer has a perimeter having at least one concave edge forming at least a portion of a boundary of the array; the at least one concave edge defines an opening between two opposing portions of the anisotropic material layer; no electrode elements are present in the opening; the anisotropic material layer has a substantially rounded V-shape comprising a first leg, a second leg, and a vertex portion connecting the first leg to the second leg, and the transducer apparatus has a shape that is the same shape or substantially same shape as the anisotropic material layer.

Illustrative Embodiment 17: The transducer apparatus of Illustrative Embodiment 16, wherein the array of one or more electrode elements comprises at least one electrode element and connecting electrically conductive material, optionally wherein the connecting electrically conductive material comprises a flexible printed circuit board (PCB).

Illustrative Embodiment 17A: The transducer apparatus of Illustrative Embodiment 17, wherein the connecting electrically conductive material comprises a flexible printed circuit board (PCB).

Illustrative Embodiment 18: The transducer apparatus of Illustrative Embodiment 17, wherein when viewed from the direction perpendicular to the front face of the anisotropic material layer, the connecting electrically conductive material comprises: a first leg portion disposed on the first leg of the anisotropic material layer; a second leg portion disposed on the second leg of the anisotropic material layer; and a vertex portion disposed on the vertex portion of the anisotropic material layer; wherein the array of one or more electrode elements comprises at least one electrode element disposed on each of the first leg portion, the second leg portion, and the vertex portion.

Illustrative Embodiment 19: The transducer apparatus of Illustrative Embodiment 16, wherein the front face of the anisotropic material layer comprises a perimeter and an interior portion; the anisotropic material layer comprises: a first slit extending from the perimeter of the front face of the anisotropic material layer towards the interior portion of the front face of the anisotropic material layer; and a second slit extending from the perimeter of the front face of the anisotropic material layer towards the interior portion of the front face of the anisotropic material layer; wherein the first slit and the second slit define the first leg portion, the second leg portion, and the vertex portion of the anisotropic material layer; and when viewed from the direction perpendicular to the front face of the anisotropic material layer, a first portion of the flexible layer extends along and covers the first slit; and a second portion of the flexible layer extends along and covers the second slit.

Illustrative Embodiment 20: The transducer apparatus of Illustrative Embodiment 16, wherein the flexible layer is a layer of foam, and wherein the layer of foam has adhesive disposed at least on a front face of the foam facing the subject's body.

Illustrative Embodiment 20A: The transducer apparatus of Illustrative Embodiment 16, wherein a thickness of the flexible layer is at least 0.1 mm and at most 5 mm.

Illustrative Embodiment 20B: The transducer apparatus of Illustrative Embodiment 19, further comprising at least a third slit disposed at one or more of an apex of the vertex portion of the anisotropic material layer and/or at a distal end opposite a vertex of at least one of the first leg portion or the second leg portion of the anisotropic material layer.

Illustrative Embodiment 20C: The transducer apparatus of Illustrative Embodiment 20B further wherein the flexible layer extends along and covers each of the slits.

Illustrative Embodiment 20D: The transducer apparatus of Illustrative Embodiment 19, further comprising: a third slit disposed at or near an apex of the vertex portion of the anisotropic material layer; a fourth slit disposed at a distal end opposite a vertex of the first leg portion of the anisotropic material layer; and fifth slit disposed at a distal end opposite a vertex of the first leg portion of the anisotropic material layer.

Illustrative Embodiment 20E: The transducer apparatus of Illustrative Embodiment 20D further wherein the flexible layer extends along and covers each of the slits.

Although the invention has been described as several example transducers and various illustrated embodiments, features of the example transducers and the illustrated embodiments may be used interchangeably together unless otherwise indicated herein or otherwise clearly contradicted by context.

Optionally, for each embodiment described herein, the transducers may be capable of being supplied with an electrical signal having an alternating current waveform at frequencies in a range from about 50 kHz to about 1 MHz, such as, for example, 100 kHz to about 300 kHz, and appropriate to deliver TTFields treatment to the subject's body. The field intensity may be from 1 V/cm to about 10 V/cm, such as from 1 V/cm to about 5 V/cm.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

Numerous modifications, alterations, and changes to the described embodiments are possible without departing from the scope of the present invention defined in the claims. It is intended that the present invention need not be limited to the described embodiments, but that it has the full scope defined by the language of the following claims, and equivalents thereof.

What is claimed is:

1. A transducer apparatus for delivering tumor treating fields to a subject's body, the transducer apparatus comprising:
an array of one or more electrode elements, the array configured to be positioned over the subject's body with a front face of the array facing the subject's body and a back face facing away from the subject's body;
an anisotropic material layer electrically coupled to the array of one or more electrode elements and located on a front side of the front face of the array, the anisotropic material layer comprising a front face and a back face, the back face facing the front face of the array; and
a non-adhesive flexible layer having a front face and a back face, the flexible layer coupled to at least a portion of the front face of the anisotropic material layer and configured to contact the subject's body optionally via a first adhesive layer disposed on the front face of the flexible layer;
wherein, when viewed from a direction perpendicular to the front face of the anisotropic material layer:
the anisotropic material layer has a perimeter having at least one concave edge forming at least a portion of a boundary of the array;
the at least one concave edge defines an opening between two opposing portions of the anisotropic material layer;
no electrode elements are present in the opening;
the anisotropic material layer has a substantially C-shaped, U-shaped, rounded V-shaped, or annular shaped surface; and
the transducer apparatus has a shape that is the same shape or substantially same shape as the anisotropic material layer.

2. The transducer apparatus of claim 1, wherein when viewed from the direction perpendicular to the front face of the anisotropic material layer:
the anisotropic material layer has a substantially C-shaped surface; and
the two opposing portions of the anisotropic material layer are curved.

3. The transducer apparatus of claim 1, wherein when viewed from the direction perpendicular to the front face of the anisotropic material layer:
the anisotropic material layer has a substantially U-shaped or rounded V-shaped surface; and
the two opposing portions of the anisotropic material layer comprise a first inner side and second inner side, respectively, which are substantially straight and extend in substantially parallel directions, or deviate from being parallel by less than 60°, wherein the first inner side borders the opening between the two opposing portions and the second inner side borders the opening between the two opposing portions and is opposite the first inner side.

4. The transducer apparatus of claim 1, wherein when viewed from the direction perpendicular to the front face of the anisotropic material layer:
the anisotropic material layer has a substantially annular shaped surface;
the opening defined by the at least one concave edge is bounded on all sides by the anisotropic material layer; and
the anisotropic material layer has a substantially circular, oval, ovaloid, ovoid, or elliptical shaped surface with the opening formed therein.

5. The transducer apparatus of claim 4, wherein the substantially circular, oval, ovaloid, ovoid, or elliptical shaped surface comprises a slit running from an exterior boundary of the anisotropic material layer to the opening allowing at least a temporary opening in the annular shape.

6. The transducer apparatus of claim 1, wherein when viewed from the direction perpendicular to the front face of the anisotropic material layer:
the first opposing portion defines a first opposing side;
the second opposing portion defines a second opposing side; and
when a straight line is drawn through the first and second opposing sides of the anisotropic material layer and across the opening, a distance along the line across the opening is at least 15% of a distance along the line across one of the opposing portions of the anisotropic material layer.

7. The transducer apparatus of claim 1, wherein
the front face of the anisotropic material layer comprises a perimeter and an interior portion;
the anisotropic material layer comprises at least one slit extending from the perimeter of the front face of the anisotropic material layer towards the interior portion of the front face of the anisotropic material layer; and
when viewed from the direction perpendicular to the front face of the anisotropic material layer, a first portion of the flexible layer extends along and covers the at least one slit.

8. The transducer apparatus of claim 7, wherein when viewed from the direction perpendicular to the front face of the anisotropic material layer, the first portion of the flexible layer further extends from a first point of the perimeter of the front face of the anisotropic material layer to a second point of the perimeter of the front face of the anisotropic material layer.

9. The transducer apparatus of claim 8, wherein when viewed from the direction perpendicular to the front face of the anisotropic material layer, the first portion of the flexible layer comprises a thicker portion covering the at least one slit and a thinner portion not covering the at least one slit.

10. The transducer apparatus of claim 7, wherein when viewed from the direction perpendicular to the front face of the anisotropic material layer, the flexible layer further comprises a second portion disposed about a perimeter of the anisotropic material layer.

11. The transducer apparatus of claim 1, wherein the anisotropic material layer comprises graphite.

12. The transducer apparatus of claim 1; wherein
the first adhesive has a first adhesive strength; and
the front face of the anisotropic material layer comprises a second adhesive having a second adhesive strength less than the first adhesive strength.

13. An assembly for applying tumor treating fields to a subject's body while avoiding at least one area with an anatomic feature or device, the assembly comprising:
a first transducer array comprising:
at least one electrode element; and
a first anisotropic material layer comprising a first surface and electrically coupled to the at least one electrode element;
wherein when viewed from a direction perpendicular to the first surface:
the first surface is substantially C-shaped, U-shaped, rounded V-shaped, or annular shaped wherein:
when C-shaped, U-shaped, or rounded V-shaped, two opposing portions of the first surface are spaced apart from each other and connected to each other by a concave edge of the first surface; and
when annular shaped, a concave edge defines an opening bounded on all sides by the first surface; and
the first transducer array has a shape that is the same shape or substantially same shape as the first surface; and
a second transducer array comprising at least one electrode element and a second anisotropic material layer, the second transducer array having a second surface comprising the same or mirror image shape, or a different shape, than the first surface, and the second transducer array has a shape that is the same shape or substantially same shape as the second surface.

14. The assembly of claim 13, wherein the first surface of the first anisotropic material layer is substantially C-shaped, U-shaped, or rounded V-shaped and is adapted to be positioned on the subject's body such that the two opposing portions of the first surface are spaced apart to straddle a first location on the subject's body coinciding with a nipple or the first location on the subject's body having a chemotherapy port, and no electrode elements of the first transducer array are located over the nipple or chemotherapy port.

15. The assembly of claim 13, wherein the first surface of the first anisotropic material layer is substantially annular shaped and is adapted to be positioned on the subject's body such that the opening coincides with a first location on the subject's body coinciding with a nipple or the first location on the subject's body having a chemotherapy port, and no electrode elements of the first transducer array are located over the nipple or chemotherapy port.

16. A transducer apparatus for delivering tumor treating fields to a subject's body, the transducer apparatus comprising:
an array of one or more electrode elements, the array configured to be positioned over the subject's body with a front face of the array facing the subject's body and a back face facing away from the subject's body;
an anisotropic material layer electrically coupled to the array and located on a front side of the front face of the array of one or more electrode elements, the anisotropic material layer comprising a front face and a back face, the back face facing the front face of the array; and
a non-adhesive flexible layer coupled to at least a portion of the front face of the anisotropic material layer and configured to contact the subject's body optionally via an adhesive layer disposed on the front face of the flexible layer;
wherein, when viewed from a direction perpendicular to the front face of the anisotropic material layer:
the anisotropic material layer has a perimeter having at least one concave edge forming at least a portion of a boundary of the array;
the at least one concave edge defines an opening between two opposing portions of the anisotropic material layer;
no electrode elements are present in the opening;
the anisotropic material layer has a substantially rounded V-shape comprising a first leg, a second leg, and a vertex portion connecting the first leg to the second leg, and
the transducer apparatus has a shape that is the same shape or substantially same shape as the anisotropic material layer.

17. The transducer apparatus of claim 16, wherein the array of one or more electrode elements comprises at least one electrode element and connecting electrically conductive material, optionally wherein the connecting electrically conductive material comprises a flexible printed circuit board (PCB).

18. The transducer apparatus of claim 17, wherein when viewed from the direction perpendicular to the front face of the anisotropic material layer, the connecting electrically conductive material comprises:
a first leg portion disposed on the first leg of the anisotropic material layer;
a second leg portion disposed on the second leg of the anisotropic material layer; and
a vertex portion disposed on the vertex portion of the anisotropic material layer;
wherein the array of one or more electrode elements comprises at least one electrode element disposed on each of the first leg portion, the second leg portion, and the vertex portion.

19. The transducer apparatus of claim 16, wherein
the front face of the anisotropic material layer comprises a perimeter and an interior portion;
the anisotropic material layer comprises:
a first slit extending from the perimeter of the front face of the anisotropic material layer towards the interior portion of the front face of the anisotropic material layer; and
a second slit extending from the perimeter of the front face of the anisotropic material layer towards the interior portion of the front face of the anisotropic material layer;
wherein the first slit and the second slit define the first leg portion, the second leg portion, and the vertex portion of the anisotropic material layer; and
when viewed from the direction perpendicular to the front face of the anisotropic material layer,
a first portion of the flexible layer extends along and covers the first slit; and
a second portion of the flexible layer extends along and covers the second slit.

20. The transducer apparatus of claim 16, wherein the flexible layer is a layer of foam, and wherein the layer of foam has adhesive disposed at least on a front face of the foam facing the subject's body.
